(19) United States
(12) Patent Application Publication
(10) Pub. No.: US 2010/0121377 A1
(43) Pub. Date: May 13, 2010

(54) LUMBAR SUPPORT DEVICE

(76) Inventor: Pamela T. Held, Charlotte, NC (US)

Correspondence Address: ADAMS INTELLECTUAL PROPERTY LAW, Suite 2350 Charlotte Plaza, 201 South College Street, CHARLOTTE, NC, 28244, US

(21) Appl. No.: 12/269,177

(22) Filed: Nov. 12, 2008

(51) Int. Cl. A61F 5/00

(52) U.S. Cl. 606/242

(57) ABSTRACT

A portable lumbar support is provided and includes an elongate resilient cushion member having a pair of opposing end sections and an intermediate section having a concave therapeutic surface on at least one major side thereof for fitting against a lumbar region of a patient, and an opposed major stabilizing surface for contacting and being supported by an adjacent support structure such as a seat back when the support is in use. Pressure from contact between the lumbar region of the patient and the support elongates a fascia portion of the lumbar region.

### TECHNICAL FIELD AND BACKGROUND OF THE INVENTION

This invention relates to a lumbar support device that includes a concave major side for fitting against a lumbar region of a patient. The support device is designed to provide elongation of the fascia tissue in the lumbar region of a patient to restore fascia tissue to its normal state. The fascia tissue of the lumbar region tends to compress from normal standing and sitting because of downward forces associated with a patient's weight compressing on the fascia tissue. This compression leads to increased slumping and patient discomfort. In addition, uneven weight distribution while sitting or standing can cause uneven compression of the fascia tissue, leading towards a more compressed fascia region on one side of the patient. Most people favor one side of their body while sitting, cause uneven weight distributed. This uneven weight distribution and compression of the fascia region can cause improper bone alignment and a compressed gastrointestinal system.

Conventional lumbar support devices are aimed at providing even distribution of forces, but do little to stop the compressive forces on the fascia region or to cause fascial elongation to normal lengths. Accordingly, these devices still leave the fascia tissue in a compressed state, causing intense patient pain and loss of flexibility.

Therefore, there is a need for a lumbar support device that is adapted to cause fascia tissue elongation. The lumbar support device may be used while sitting and prevents slumping or twisting of a patient.

### SUMMARY OF THE INVENTION

Therefore, it is an object of the invention to provide a lumbar support device for supporting the lumbar area of a patient.

It is another object of the invention to provide a lumbar support device for elongating the fascia tissue of the lumbar area of a patient.

It is another object of the invention to provide a lumbar support device for maintaining a patient in an upright position.

It is another object of the invention to provide a lumbar support device for causing even distribution of forces on the lumbar area of a patient.

These and other objects and advantages are achieved by providing a portable lumbar support. The support includes an elongate resilient cushion member having a pair of opposing end sections and an intermediate section having a concave therapeutic surface on at least one major side thereof for fitting against a lumbar region of a patient, and an opposed major stabilizing surface for contacting and being supported by an adjacent support structure such as a seat back when the support is in use. Pressure from contact between the lumbar region of the patient and the support elongates a fascia portion of the lumbar region.

According to another embodiment of the present invention, the opposing end sections are generally rounded in cross-section.

According to another embodiment of the present invention, the intermediate mid section is generally non-rounded in cross-section.

According to another embodiment of the present invention, the opposing end sections are generally rounded in cross-section and the intermediate mid section is generally non-rounded in cross-section.

According to another embodiment of the present invention, the overall length of the support is between about 17 and about 22 inches, and the length of intermediate mid section is between about 4 and about 22 inches.

According to another embodiment of the present invention, the support is fabricated of an envelope filled with a granular material.

According to another embodiment of the present invention, the intermediate mid section has a thickness of between about 25 and 75 percent of the thickness of the opposing end sections.

According to another embodiment of the present invention, the opposing end sections and the intermediate section are integrally formed.

According to another embodiment of the present invention, each end section of the pair of opposing end sections tapers inward.

According to another embodiment of the present invention, the resilient cushion member is formed from at least three cushion layers laminated onto one another.

According to another embodiment of the present invention, the resilient cushion member comprises a plurality of deformable chambers, each deformable chamber of the plurality of deformable chambers having a valve for adjusting pressure within each deformable chamber.

According to another embodiment of the present invention, the support also includes a plurality of gripping members for attaching to an exterior of the support and providing resistance to sliding of the portable lumbar support.

According to another embodiment of the present invention, the support is fabricated of a foam material.

According to another embodiment of the present invention, the foam comprises polyethylene, closed cell foam, dry fast foam, memory foam, or combinations thereof.

According to another embodiment of the present invention, the support also includes a cover for covering the lumbar support.

According to another embodiment of the present invention, the support is fabricated from products that are for heating or cooling for increased therapeutic value, the products comprising pot barley, buckwheat, seeds, pits, herbs, spices, or combinations thereof.

In another embodiment of the present invention, a portable lumbar support for alleviating pain associated with compression of a fascia region of a lumbar portion of a patient is provided. The support includes an elongate cushion member having a pair of opposing end sections and an intermediate section having a thickness less than a thickness of each end section of the pair of opposing end sections and having a surface on at least one major side thereof for fitting against the lumbar region of the patient, and an opposed major stabilizing surface for contacting and being supported by an adjacent support structure such as a seat back when the support is in use, wherein a pressure point caused by contact between each end section of the pair of opposing end sections and the lumbar portion of the patient causes elongation of the fascia region.

### BRIEF DESCRIPTION OF THE DRAWINGS

Some of the objects of the invention have been set forth above. Other objects and advantages of the invention will appear as the description of the invention proceeds when taken in conjunction with the following drawings, in which:

FIG. 1 is a perspective view of a lumbar support device according to the present invention;

FIG. 2 is a side view of a lumbar support device according to an alternate embodiment of the present invention;

FIG. 3 is a side view of a lumbar support device according to an alternate embodiment of the present invention;

FIG. 4 is a side view of a lumbar support device according to an alternate embodiment of the present invention;

FIG. 5 is a side view of a lumbar support device according to an alternate embodiment of the present invention;

FIG. 6 is a side view of a lumbar support device according to an alternate embodiment of the present invention;

FIG. 7 is a side view of a lumbar support device according to an alternate embodiment of the present invention;

FIG. 8 is a side view of a lumbar support device according to an alternate embodiment of the present invention;

FIG. 9 is a rear view of a patient wearing the lumbar support device according to the present invention;

FIG. 10 is a perspective view of a patient wearing the lumbar support device against a nearby structure; and

FIG. 11 is an anatomical rear view of a patient wearing the lumbar support device according to the present invention.

### DESCRIPTION OF THE PREFERRED EMBODIMENTS AND BEST MODE

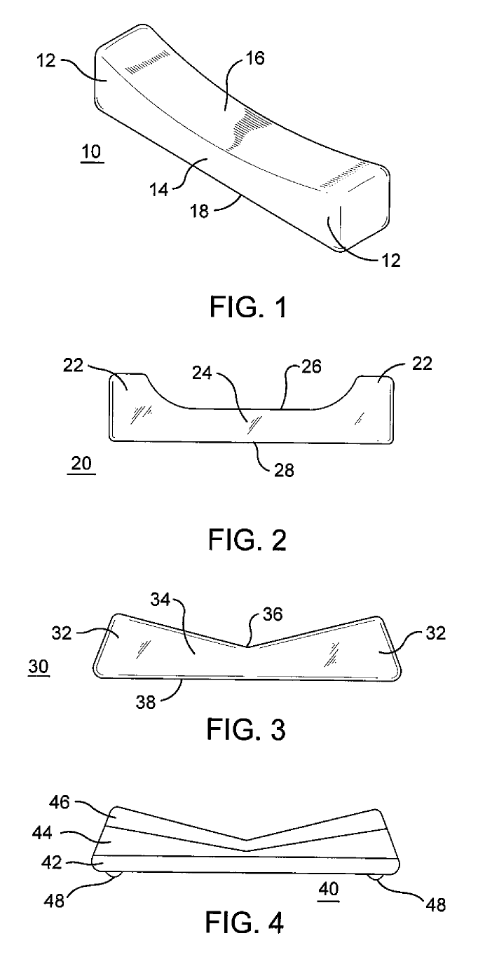

Referring now specifically to the drawings, a portable lumbar support device according to the present invention is shown generally in FIG. 1 at reference numeral 10. The support 10 includes a pair of opposing end sections 12 and an intermediate section 14. The intermediate section 14 will generally have a concave therapeutic surface 16. The therapeutic surface 16 is designed for fitting against the lumbar region of a patient's back. A substantially flat surface 18 is provided on an opposing side from therapeutic surface 16 and is adapted for fitting against a support structure such as a chair back, wall, or bed. In this manner, contact between the lumbar support 10 and the support structure creates pressure against the lumbar region of the patient.

An alternate embodiment of the present invention is shown in FIG. 2. In this embodiment, lumbar support device 20 includes a pair of opposing end sections 22 and an intermediate section 24. The intermediate section 24 will generally have a concave therapeutic surface 26. The therapeutic surface 26 is designed for fitting against the lumbar region of a patient's back. A substantially flat surface 28 is provided on an opposing side from therapeutic surface 26 and is adapted for fitting against a support structure such as a chair back, wall, or bed. The opposing end sections 22 are generally square in this embodiment.

An alternate embodiment of the present invention is shown in FIG. 3. In this embodiment, lumbar support device 30 includes a pair of opposing end sections 32 and an intermediate section 34. The intermediate section 34 will generally have a concave therapeutic surface 36. The therapeutic surface 36 is designed for fitting against the lumbar region of a patient's back. A substantially flat surface 38 is provided on an opposing side from therapeutic surface 36 and is adapted for fitting against a support structure such as a chair back, wall, or bed. The opposing end sections 32 generally taper inwards in this embodiment. The therapeutic surface 36 is formed from a pair of tapering surfaces that combine at about the center of therapeutic surface 36.

An alternate embodiment of the present invention is shown in FIG. 4. In this embodiment, lumbar support device 40 has the same perimeter structure as lumbar support device 30. However, lumbar support device 40 is formed from a plurality of layers 42, 44, and 46. These layers 42, 44, and 46 may be made of 100% polyethylene foam with a density of 2.3 to 4.0 pounds and closed cell foam structure and may be laminated or glued onto one another. A plurality of gripping surfaces 48 may be provided on the surfaces of the lumbar support device 40 to improve holding capabilities of the support device 40 against a structure.

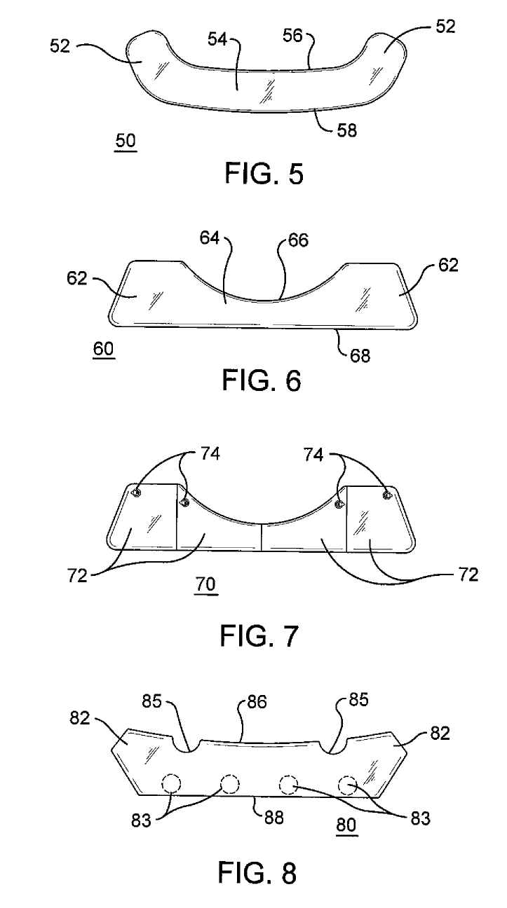

An alternate embodiment of the present invention is shown in FIG. 5. In this embodiment, lumbar support device 50 includes a pair of opposing end sections 52 and an intermediate section 54. The intermediate section 54 will generally have a concave therapeutic surface 56. The therapeutic surface 56 is designed for fitting against the lumbar region of a patient's back and is generally concaved in shape. An arcuate surface 58 is provided on an opposing side from therapeutic surface 56 and is adapted for fitting against a support structure such as a chair back, wall, or bed. The opposing end sections 52 are generally rounded in cross-section in this embodiment. This allows for increased rollover ability of the patient while wearing the lumbar support device 50.

An alternate embodiment of the present invention is shown in FIG. 6. In this embodiment, lumbar support device 60 includes a pair of opposing end sections 62 and an intermediate section 64. The intermediate section 64 will generally have a concave therapeutic surface 66. The therapeutic surface 66 is designed for fitting against the lumbar region of a patient's back and is generally concaved in shape. A substantially flat surface 68 is provided on an opposing side from therapeutic surface 66 and is adapted for fitting against a support structure such as a chair back, wall, or bed. The opposing end sections 62 are generally trapezoidal shaped in cross-section in this embodiment. This allows for increased stability and roll-over inhibition of the patient while wearing the lumbar support device 60.

An alternate embodiment of the present invention is shown in FIG. 7. In this embodiment, lumbar support device 70 has the same perimeter structure as lumbar support device 60. However, lumbar support device 70 includes a plurality of deformable chambers 72 and fluid valves 74 for allowing fluid to enter and exit the deformable chambers. In this manner, the deformable chambers can be adjusted for various pressure setting depending on the patient's needs and desires.

An alternate embodiment of the present invention is shown in FIG. 8. In this embodiment, lumbar support device 80 includes a pair of opposing end sections 82 and a concave therapeutic surface 86 of at least 5 inches. The therapeutic surface 86 is designed for fitting against the lumbar region of a patient's back and is generally concaved in shape. An arcuate surface 88 is provided on an opposing side from therapeutic surface 86 and is adapted for fitting against a support structure such as a chair back, wall, or bed. The opposing end sections 82 are generally rounded in cross-section in this embodiment. This allows for increased rollover ability of the patient while wearing the lumbar support device 80. A plurality of intensifiers 83 may be placed within the lumbar support device 80 to increase elongation of the fascia caused by the lumbar support device 80. The intensifiers 83 will preferably be spherical plastic or wooden objects having a diameter of approximately at least one inch, and act to create intense pressure points about the patient. A plurality of cutouts 85 are defined on the therapeutic surface 86 and are preferably at least one inch in diameter and for providing increase curvature and fit of the device 80.

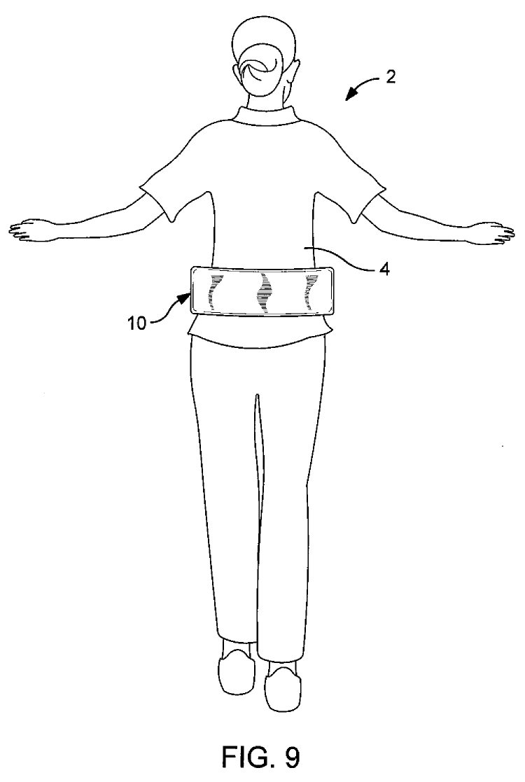

Application of the lumbar support device 10 is shown in FIGS. 9-11. It shall be appreciated that while lumbar support device 10 is shown in FIGS. 9-11, any of the lumbar support devices discussed in regards to FIGS. 1 through 8 may be employed. The lumbar support device 10 is placed against the lumbar region 4 of a patient 2. In preferred embodiments, the generally concave therapeutic surface 16 will be placed against the lumbar region 4.

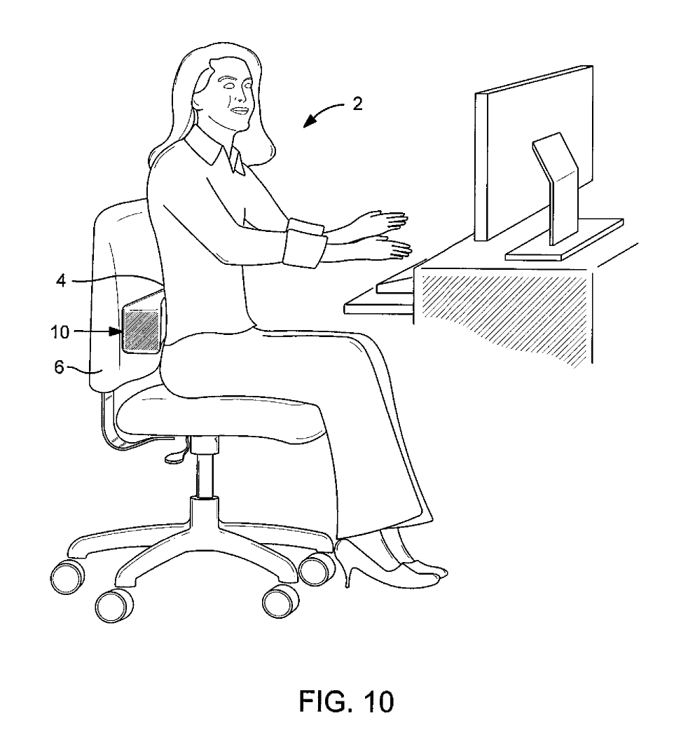

As shown in FIG. 10, the lumbar support device 10 is placed between a structure, in this case a chair 6, and the lumbar region 4 of the patient 2. The lumbar support device 10 can be positioned in a variety of vertical locations as long as the support is positioned against the lumber region 4 of the patient 2. The lumbar support device 10 will generally be positioned within the horizontal center of the lumbar region 4 of the patient 2. In this manner, this will create symmetry of the therapeutic surface 16 against the lumbar region 4.

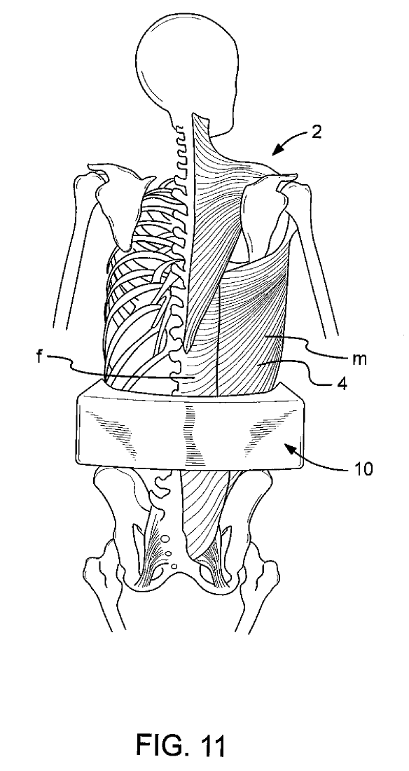

FIG. 11 shows an anatomical view of the lumbar support device 10 positioned against the lumbar region 4 of the patient 2. As shown in this view, the fascia region f and muscular region m of the lumbar region 4 are shown in the anatomical view. Upon routine daily activities, the fascia region f and muscular region m are compressed by the patient's weight. The lumbar support device 10 acts to elongate the compressed regions of fascia f and muscle m and to maintain the patient 2 is a symmetric position.

In some embodiments, therapeutic surface 16 works as the stabilizing surface and flat surface 18 is the therapeutic surface. Upon compression by the patient, the opposing end sections 12 will create pressure against the lumbar region 4 of the patient 2 and work to elongate the compressed fascia region f and muscular region m. It is appreciated that the shape of the pair of end sections will be determined based on individual patient characteristics and desires.

In other embodiments, horizontal or vertical channels are provided to increase drying capabilities and compression ratios of the support device 10. Still in other embodiments, a phifertex fabric is employed as a cover. Memory foam having a density of 4.3 to 5 pounds is employed in other embodiments, and still yet, in additional embodiments, an attachment trap is employed. Still yet, in additional embodiments, a natural product such as pot barley, buckwheat, seeds, pits, herbs, spices, or combinations thereof are employed. Polyethylene, closed cell foam, dry fast foam, memory foam, or combinations thereof are employed.

In the preferred embodiment, the support device 10 has a length between about 17 and 22 inches, a length of the intermediate mid section of between about 4 and 22 inches. Opposing end sections have a width of between about 1 inches and 8 inches. The intermediate mid section has a thickness of between about 25 percent and 75 percent of the thickness of the opposing end sections. The lumbar support device 10 is preferably fabricated from a molded or extruded process. The support device 10 is made from polyethylene closed cell foam having a density of 2.3 to 4.0 pounds. A thin strip of fiberglass, plastic, copolymer resin, or kevlar® may be used to prevent tearing and also provide waterproof characteristics. This thin strip can be best described as a structural stabilizer and can be used in any of the embodiments described herein. This thin strip may also provide a rigid side and a soft side for comfort.

A lumbar support device is described above. Various details of the invention may be changed without departing from its scope. Furthermore, the foregoing description of the preferred embodiment of the invention and the best mode for practicing the invention are provided for the purpose of illustration only and not for the purpose of limitation—the invention being defined by the claims.

We claim:

1. A portable lumbar support, comprising an elongate resilient cushion member having a pair of opposing end sections and an intermediate section having a concave therapeutic surface on at least one major side thereof for fitting against a lumbar region of a patient, and an opposed major stabilizing surface for contacting and being supported by an adjacent support structure such as a seat back when the support is in use, wherein pressure from contact between the lumbar region of the patient and the support elongates a fascial portion of the lumbar region.

2. A portable lumbar support according to claim 1, wherein the opposing end sections are generally rounded in cross-section.

3. A portable lumbar support according to claim 1, wherein the intermediate mid section is generally non-rounded in cross-section.

4. A portable lumbar support according to claim 1, wherein the opposing end sections are generally rounded in cross-section and the intermediate mid section is generally non-rounded in cross-section.

5. A portable lumbar support according to claim 1, wherein the overall length of the support is between about 17 and about 22 inches, and the length of intermediate mid section is between about 4 and abut 22 inches.

6. A portable lumbar support according to claim 1, wherein the support is fabricated of an envelope filled with a granular material.

7. A portable lumbar support according to claim 4, wherein the intermediate mid section has a thickness of between about 25 and 75 percent of the thickness of the opposing end sections.

8. A portable lumbar support according to claim 1, wherein opposing end sections and the intermediate section are integrally formed.

9. A portable lumbar support according to claim 1, wherein each end section of the pair of opposing end sections tapers inward.

10. A portable lumbar support according to claim 1, wherein the resilient cushion member is formed from at least three cushion layers laminated onto one another.

11. A portable lumbar support according to claim 1, wherein the resilient cushion member comprises a plurality of deformable chambers, each deformable chamber of the plurality of deformable chambers having a valve for adjusting pressure within each deformable chamber.

12. A portable lumbar support according to claim 1, further comprising a plurality of gripping members for attaching to an exterior of the support and providing resistance to sliding of the portable lumbar support.

13. A portable lumbar support according to claim 1, wherein the support is fabricated of a foam material.

14. A portable lumbar support according to claim 13, wherein the foam comprises polyethylene, closed cell foam, dry fast foam, memory foam, or combinations thereof.

15. A portable lumbar support according to claim 1, further including a cover for covering the lumbar support.

16. A portable lumbar support according to claim 1, wherein the support is fabricated from products that are for heating or cooling for increased therapeutic value, the products comprising pot barley, buckwheat, seeds, pits, herbs, spices, or combinations thereof.

17. A portable lumbar support for alleviating pain associated with compression of a fascial region of a lumbar portion of a patient, comprising an elongate cushion member having a pair of opposing end sections and an intermediate section having a thickness less than a thickness of each end section of the pair of opposing end sections and having a surface on at least one major side thereof for fitting against the lumbar region of the patient, and an opposed major stabilizing surface for contacting and being supported by an adjacent support structure such as a seat back when the support is in use, wherein a pressure point caused by contact between each end section of the pair of opposing end sections and the lumbar portion of the patient causes elongation of the fascial region.
